(19) United States
(12) Patent Application Publication
(10) Pub. No.: US 2006/0134099 A1
(43) Pub. Date: Jun. 22, 2006

(54) Production of rabies antibodies in plants

(75) Inventors: Hilary Koprowski, Wynnewood, PA (US); Kisung Ko, Drexel Hill, PA (US); Pauline M. Rudd, Oxford (GB); Raymond A. Dwek, Oxford (GB); Yoram Tekoah, Beer Sheva (IL)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: BIOTECHNOLOGY FOUNDATION, INC., Philadelphia, PA (US); UNIVERSITY OF OXFPRD, Oxford (GB)

(21) Appl. No.: 10/559,236

(22) PCT Filed: Jun. 2, 2004
(86) PCT No.: PCT/US04/17544

§ 371 Date: Feb. 14, 2006

Related U.S. Application Data
(60) Provisional application No. 60/475,376, filed on Jun. 2, 2003.

(51) Int. Cl. A61K 39/395

(52) U.S. Cl. 424/132.1; 530/387.1; 435/320.1; 800/298

(57) ABSTRACT

Human rabies monoclonal antibodies (rabies MAb^{P}) comprising rabies human MAb S057 heavy chain and light chain subunits are expressed and assembled in plants under the control of two strong constitutive promoters. Additionally, regulatory control elements such as alfalfa mosaic virus untranslated leader sequence and Lys-Asp-Glu-Leu (KDEL) endoplasmic reticulum retention signal were linked at the N- and C-terminus of the heavy chain of human rabies MAb^{P}, respectively to regulate expression of the rabies MAb^{P}. Rabies MAb^{P} was as effective at neutralizing the activity of the rabies virus as the mammalian-derived antibody (MAb^{M}) or human rabies immunoglobulin (HRIG).

### I. FIELD OF THE INVENTION

The invention is directed to immunological compositions and methods of making and using same. In particular, the invention is directed to plant-derived antibodies and their use as therapeutic agents against viral infections in humans.

### II. BACKGROUND OF THE INVETION

Transgenic plants have proven to be an efficient production system for the expression of functional therapeutic proteins (Daniell et al. (2001) Trends. Plant Sci. 6:219-26), The high cost of production and purification of synthetic peptides manufactured by chemical or fermentation based processes may prevent their broad scale use as therapeutics. The production of therapeutic proteins in transgenic plants offer an economical alternative.

Plant-derived monoclonal antibodies (MAb^{P}) have several advantages as compared to their mammalian-derived counterparts, namely, the lack of animal pathogenic contaminants, low cost of production, and ease of agricultural scale-up as compared to the conventional fermentation methods. Since the initial report of functional MAbs expressed in transgenic plants (Hiatt et al. (1989) Nature 342:76-78), therapeutic and diagnostic MAb^{P}s have been successfully produced in transgenic tobacco, soybean, alfalfa (Ma et al. (1998) Nat. Med. 4:601-6; Zeitlin et al.(1998) Nat. Biotechnol. 16:1361-4; Khoudi et al. (1999) Biotechnol. Bioeng. 64:13543; Bouquin et al. (2002) Transgenic Res. 11:115-22) and other plants (Daniell et al., supra). Two MAbs^{P} have recently been used for topical passive immunization against Streptococcus mutans and herpes simplex virus in animals (Ma et al., supra; Zeitlin et al., supra). The expression level and appropriate posttranslational events, for example, correct folding, glycosylation, subcellular targeting, are important factors for the effectiveness of antibodies produced in plants (Khoudi et at, supra; Mann et at (2003) Nat. Biotechnol. 21:255-261; Sharp et al. (2001) Bioteclinol. Bioeng. 73:338-346; Jobling et al. (2003) Nat. Biotechnol. 21:77-80). Differences in post-translational modifications, such as glycosylation, have been shown to influence the properties of plant-derived proteins (Daniell et al, supra; Conrad et al (1998) Plant Mol. Biol. 38:101-109; Mann et at (2003) Nat. Biltechnol. 21:255-261). In plants, N-linked glycans may contain antigenic (Faye et at (1993) Anal. Biochem. 109:104-108) and/or allergenic (van Ree et at (2000) J. Biol. Chem. 275:11451-11458) β(1,2)-xylose (Xyl) residues attached to the N-linked Mannose of the glycan core and α(1,3)-fucose (Fuc) residues linked to the proximal GlcNAc that are not present on mammalian glycans. Plant glycans, however, do not contain sialic acid residues and plant antibodies do not require these residues for successful topical passive immunization (Ma et al., supra; Zeitlin et al., supra).

Glycosylation processing in the endoplasmic reticulum (ER) is conserved amongst almost all species and restricted to oligomannose (Man₅₋₉GlcNAc₂) type N-glycans, whereas the Golgi-generated processing to hybrid and complex type glycans is highly diverse (Helenius et al (2001) Science 291:2364-2369). When attached to the C-terminus, the ER retrieval motif, KDEL, allows glycoproteins to be retained in, or returned to, the ER. Although there are exceptions (Navazio et al. (2002) Biochemistry 41:14141-14149), in general glycans attached to proteins containing a C-terminal KDEL sequence would be expected to be restricted mainly to the oligomannose type N-glycans (Helenius et al. (2001) Science 291:2364-2369; Henderson et al. (1997) Planta 202:313-323; Bauly et al. (2000) Plant Physiol. 124:1229-1238).

ER retention of expressed proteins in transgenic plants usually improves the production levels (Conrad et al. (1998) Plant Mol. Biol. 38: 101-109; Sharp et al. (2001) Biotechnol. Bioeng. 73:338-346). However, since glycan processing can affect the stability of antibodies (Rudd et al. (2001) Science 291:2370-2376), it is unclear whether a MAb^{P} with modified glycan structures would be active and able to confer effective systemic post-exposure prophylaxis.

The invention, as disclosed and described herein, overcomes the prior art problems with plant-derived antibodies by optimizing factors related to gene regulatory elements in plants and stable expression of monoclonal antibodies in transgenic plants. The invention provides methods and compositions for the production of antiviral plant-derived antibodies for passive or active immunization against viral infections.

### III. SUMMARY OF THE INVENTION

The invention, as disclosed and described herein, provides methods and compositions for detecting, treating, preventing, or ameliorating a viral disease or disorder in a mammal, inclusive of humans.

In one aspect, the invention provides a plant-derived human monoclonal antibody comprising a rabies MAb^{P}, wherein the rabies MAb^{P} contains predominantly oligomannose type N-glycans and has substantially reduced and preferably no α(1,3)-linked fucose residues. Substantially reduced α(1,3)-linked fucose residues refers to a concentration range of about 10% to 0% of α(1,3)-linked fucose residues, for example, about 8%, 6%, 4%, 2% of α(1,3)-linked fucose residues.

In one embodiment, the plant-derived human monoclonal antibody is derived from rabies human MAb S057. In another embodiment, rabies MAb^{P} is encoded by a polynucleotide molecule comprising SEQ ID NO: 1, SEQ ID NO: 3, or a combination thereof, or a polynucleotide molecule having a sequence that is substantially homologous to SEQ ID NO: 1, SEQ ID NO: 3, or a combination thereof. SEQ ID NO: 1 encodes an antibody heavy chain. SEQ ID NO: 3 encodes an antibody light chain.

In yet another embodiment, the rabies MAb^{P} comprises a polypeptide molecule comprising SEQ ID NO: 2, SEQ ID NO: 4, or a combination thereof, or a polypeptide molecule having a sequence that is substantially homologous to SEQ ID NO: 2, SEQ ID NO: 4, or a combination thereof. S

In another aspect, the invention provides an expression vector comprising, one or more gene constructs comprising polynucleotides encoding one or more rabies human MAb S057 subunits under the control of one or more promoters, operatively linked to regulatory control elements and Agrobacterium T-DNA terminal repeats.

In one embodiment, the regulatory control elements comprise an alfalfa mosaic virus untranslated leader sequence, an endoplasmic reticulum retention signal, or both. In a preferred embodiment, the rabies human MAb S057 subunits comprise a heavy chain, a light chain, or both. In a more preferred embodiment, the alfalfa mosaic virus untranslated leader sequence and endoplasmic reticulum retention signal were linked at the N- and C-terminus of the heavy chain, respectively.

In yet another embodiment, the heavy chain, the light chain or both are under one or more promoters. In a preferred embodiment, the one or more promoters comprise one or more constitutive promoters. In a more preferred embodiment, the constitutive promoters comprise a cauliflower mosaic virus 35S promoter with duplicated upstream B domains, and a potato proteinase inhibitor II promoter.

In a preferred embodiment, the expression vector is pBIRA-57.

In yet another aspect, the invention provides transgenic plants comprising the expression vectors of the invention. In a preferred embodiment, the transgenic plant comprises pBIRA-57 expression vector. In a more preferred embodiment, the transgenic plant is a tobacco plant comprising whole plant, plant cells, tissues, and organs.

In another aspect, the invention provides a pharmaceutical composition for treating, ameliorating, preventing, or detecting a rabies virus related disease or disorder comprising a pharmaceutically effective amount of a rabies MAb^{P}, and an acceptable carrier or diluent.

In yet another aspect, the invention provides a vaccine composition to induce passive immunity against rabies infection in humans comprising a rabies MAb^{P} S057, and an adjuvant.

In a further aspect of the invention, there is provided a diagnostic test kit for detection of rabies infection comprising a rabies MAb^{P} and a detection agent comprising a detectable label.

These and other aspects of the invention are disclosed in detail herein.

### IV. BRIEF DESCRIPTION OF THE FIGURES

FIG. 1. Generation of transgenic tobacco plants expressing human rabies MAb^{P}. (A) Schematic diagram of human MAb S057 light chain (LC) and heavy chain (HC) arrangement in a binary vector (pBIRA-57) used for plant transformation. LC and modified HC were placed co-directionally under the control of the Pin2 promoter (Pin2p) and CaMV35S promoter (Ca2p), respectively. AMV is alfalfa mosaic virus untranslated leader sequence of RNA 4, and KDEL is the 3′ endoplasmic reticulum (ER) retention motif. T¹ is the Pin2 terminator and T² is the NOS3 terminator. (B) PCR analysis of independent transgenic lines (#1-14) for the presence of both MAb S057 HC and LC in the plant genomic DNA. WT stands for wild type plant. (C) ELISA analysis of the same plant transgenic lines for MAb S057 protein expression. (D) Transgenic plant with high level of human MAb S057 expression (line R8) compared to the wild type tobacco plant (WT).

FIG. 2. Analysis of MAb^{P} protein expression in transgenic plants by SDS-PAGE. (A) Heavy chain (HC, 50 kD) and light chain (LC, 25 kD) of mammalian-derived MAb (MAbM) and total protein of R8 were resolved on the gel under denaturing conditions and either stained (left panel) or blotted for further detection with goat anti-human Fc_{γ} (middle panel) or F(ab′)₂ (right panel) specific antibody conjugated with horseradish peroxidase. Purified MAbM was loaded at 30 ng per lane. Soluble proteins from leaf tissue of WT and transgenic plant R8 were loaded at 40 μg per lane. Asterisks and diamond in the middle and right panels indicate additional bands recognized by Fc_{γ} or F(ab′)₂ antibodies, respectively. (B) Affnity-purified MAbs from plant and mammalian expression systems were resolved by SDS-PAGE at 3.5 μg and 2.5 μg per lane, respectively.

FIG. 3. NP-HPLC chromatograms of 2-AB4abeled N-glycans released enzymatically with PNGase F from heavy chains of plant-derived MAb (MAb^{P}) and manmalian-derived MAb (MAb^{M}). The schematic glycan structures of the main peaks found in N-glycan pools from MAb^{P} and MAb^{M} are indicated in the top and bottom panels, respectively. All peaks were numbered and corresponding glycans were assigned using an array of exoglycosidase enzymes and confirmed by MALDI-TOF mass spectrometry as explained in the examples. The symbols of the glycan structures are as follows: black square, GlcNAc; clear circle, mannose; clear diamond, galactose; diamond with a dot inside, fucose; black star, sialic acid; clear triangle, xylose; dotted line, α-linkage; solid line, β-linkage; |, 1-2 linkage; /, 1-3 linkage; -, 14 linkage; \, 1-6 linkage; ˜, undetermined linkage.

FIG. 4. Stability of plant- and mammalian- derived MAb protein in mice: The duration of MAb was determined by ELISA for the presence of antibody in serum from BALB/c mice injected intraperitoneally with MAb^{P} or MAb^{M}. Samples were collected over a period of 10 days after injection and analyzed against CVS-11 rabies virus strain.

### V. DETAILED DESCRIPTION OF THE INVENTION

The invention, as disclosed and described herein, provides compositions and methods for the detection, treatment, prevention and/or amelioration of viral infections. In particular, the invention disclosed herein demonstrates production of anti-rabies monoclonal antibodies in plants having a high yield and immunogenicity while exhibiting reduced allergenic epitopes. The invention further demonstrates the feasibility of manipulation of gene construct arrangements to express and assemble functional anti-rabies monoclonal antibodies in plants. The expressed antibody fragments of the invention fully assembled in plants without any gene silencing through several generations of the transgenic lines that maintained and expressed antibody genes.

In one aspect, the invention provides anti-rabies monoclonal antibodies in plants through the use of plant expression vectors containing one or more T-DNA constructs harboring polynucleotides encoding antibody genes placed under at least one inducible promoter and/or at least one constitutive promoter.

In a preferred embodiment, the polynucleotides encode light chain (LC) and/or heavy chain (HC) of a human derived rabies monoclonal antibody. The polynucleotides encoding LC, HC or both are placed under one or more different or the same promoters comprising inducible promoters, constitutive promoters, or both. In a more preferred embodiment, polynucleotides encoding LC and HC are placed under constitutive promoters comprising potato proteinase inhibitor II (pin 2p) and constitutive duplicated CaMV 35S promoter (Ca2p), respectively.

The immunogenicity of the plant-derived monoclonal antibodies of the invention was comparable to their mammalian-derived counterpart. The plant-derived monoclonal antibodies exhibited a similar neutralizing activity against a cell culture adapted virus as compared to their counterpart mammalian cell culture-derived monoclonal antibodies, while they exhibited a higher activity against street viruses.

According to another aspect, the invention provides plant expression vectors carrying the gene constructs of the invention. The gene constructs of the invention comprise polynucleotides encoding antibody or antibody subunit genes, promoters and regulatory control elements. The regulatory control elements are operably linked to polynucleotides encoding antibody genes. The function of the regulatory control elements, by way of example and not limitation, includes avoiding homology-based gene silencing, increasing HC and LC gene expression levels, and inducing compartment-specific accumulation, among others.

In one embodiment, the regulatory control elements are operably linked to polynucleotides encoding LC, HC or both. In another embodiment, the regulatory control elements are operably linked to polynucleotides encoding HC. In a preferred embodiment, the regulatory control elements comprise a translation alfalfa mosaic virus untranslated leader sequence AMV activator, an ER retention signal KDEL, or both.

### DEFINITIONS

The definitions used in this application are for illustrative purposes and do not limit the scope of the invention.

As used herein, the term “plant” refers to whole plants, plant organs (i.e., leaves, stems, flowers, roots, etc.), seeds and plant cells (including tissue culture cells), and progeny of same. The class of plants that can be used in the method of the invention is generally as broad as the class of higher plants amenable to transformation techniques, including both monocotyledonous and dicotyledonous plants, as well as certain lower plants such as algae. Suitable plants include plants of a variety of ploidy levels, including polyploid, diploid and haploid. The term “transgenic plant” refers to a plant modified to express one or more antibody genes.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in plant cells; (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into a plant expression vector.

As used herein “promoter” refers to a region of a DNA sequence active in the initiation and regulation of the expression of a structural gene. This sequence of DNA, usually upstream to the coding sequence of a structural gene, controls the expression of the coding region by providing the recognition for RNA polymerase and/or other elements required for transcription to start at the correct site.

As used herein, “protein” is used interchangeably with polypeptide, peptide and peptide fragments.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit protective immunity.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

As disclosed herein, “substantially homologous sequences” include those sequences which have at least about 50%, homology, preferably at least about 60%, more preferably at least about 70% homology, even more preferably at least about 80% homology, and most preferably at least about 95% or more homology to the polynucleotides of the invention.

As used herein “vaccine” refers to compositions that result in both active and passive immunizations. Both polynucleotides and their expressed gene products are referred to as vaccines herein.

As used herein “polypeptides” include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, homologous polypeptides, oligopeptide, homodimers, heterodimers, variants of polypeptides, modified polypeptides, derivatives, analogs, fusion proteins, agonists, antagonists, or antibody of the polypeptide, among others. The polypeptides include natural peptides, recombinant peptides, synthetic peptides, or a combination thereof.

As used herein, “antibody” refers to intact molecules as well as to fragments thereof, such as Fab, F(ab′)₂, and Fv fragments, which are capable of binding an epitopic determinant. Antibody fragments refer to antigen-binding immunoglobulin peptides which are at least about 5 to about 15 amino acids or more in length, and which retain some biological activity or immunological activity of an immunoglobulin.

As used herein “monoclonal antibody” refers to antibodies which display a single binding specificity and affinity for a particular epitope. Preferably, these antibodies are mammalian antibodies, including murine, human and humanized antibodies. The term “human monoclonal antibody” as used herein, refers to antibodies displaying a single binding specificity which have variable and constant regions derived from human germline immunoglobulin sequences.

1. Rabies MAb^{P}

According to one aspect, the invention provides compositions and methods for the detection, treatment, prevention and/or amelioration of lyssavirus infections, specifically rabies infections, in mammals including human. Rabies virus-specific antibodies are essential for the post-exposure rabies prophylaxis. Currently used antibodies from human serum (human anti-rabies immunoglobulin (HIRG)) or immunized horses (equine anti-rabies immunoglobulin (ERIG)) have drawbacks of safety and cost Plant-derived rabies MAb^{P} of this invention provides an economically feasible and clinically superior alternative for the passive immunization against rabies virus.

Rabies MAb^{P} of the invention exhibited immunogenicity and antigenicity in vivo, as compared to its mammalian counterpart human MAb S057 (MAb^{M}) expressed in murine/human hybridoma cell lines (Dietzschold et al., J. Virol. 64:3087-3090 (1990), incorporated herein by reference in its entirety) and/or commercial HRIG. Rabies MAb^{P} exhibited elevated rabies virus neutralization activity, protein stability, and N-glycan processing, and demonstrated efficacy in rabies virus post-exposure prophylaxis in exposed animals.

MAb^{P}s of the invention exhibited structural differences as compared to their mammalian-derived counterpart. Structural differences in proteins expressed in heterologous systems are known to arise from posttranslational modifications, mostly from glycosylation. In order to compare structural differences between plant-derived and hybridoma-derived monoclonal antibodies, heavy chains of rabies MAb^{P} and MAb^{M} S057 were isolated on SDS-PAGE and the glycans were released directly from the gel bands with two different endoglycosidases, namely, peptide N-glycosidase F (PNGase F), which releases N-glycans that do not contain an α(1,3)-Fuc residue linked to the core GlcNAc proximal to the Asn residue, and peptide N-glycosidase A (PNGase A), which liberates all N-glycans.

Human MAb S057 has a conserved N-linked glycosylation site in each HC. Rabies MAb^{P} contained predominantly oligomannose type N-glycans and had substantially reduced or no potentially antigenic α(1,3)-linked fucose residues. Rabies MAb^{P} had a shorter half-life than MAb^{M} and was as efficient as HRIG for post-exposure prophylaxis against rabies virus in hamsters, indicating that differences in N-glycosylation do not affect the efficacy of the antibody in this model.

These results demonstrate that rabies MAb^{P}, containing predominantly oligomannose type N-glycans, has anti-rabies virus neutralizing activity comparable to that of its mammalian-derived counterpart, and an efficacy in rabies post-exposure prophyalxis comparable to that of HRIG.

In one embodiment, rabies MAb^{P} was modified to contain a KDEL sequence. In contrast to MAb^{M} which contains 17 complex N-glycans in the conserved glycosylation sites on heavy chains, rabies MAb^{P}, modified to contain a KDEL sequence, displays predominantly oligomannose type N-glycans, for example, about 70%, 80%, 90%, 95% or more oligomannose type N-glycans were identified. A previous report indicated that MAb^{P} (lacking an ER retention signal) contains a greater diversity of N-glycan structures (Cabanes-Macheteau et al. (1999) Glycobiology 9:365-72) incorporated by reference herein.

The presence of Man₆₋₉GlcNAc₂ (about 70-95%, preferably 90%), GlcNAc₂Man₃GlcNAc₂ (about 3-6% preferably about 4.3%) and GlcNAc₂(Xyl)Man₃GlcNAc₂ (about 3-7%, preferably about 5.7%) glycans in MAb^{P} indicates that most of MAb^{P}/KDEL did not pass further along the secretory pathway than the cis-Golgi stack, from which it was probably retrieved and returned to the ER (Henderson et al. (1997) Planta 202:313-23; and Bauly et al.(2000) Plant Physiol. 124:1229-1238, each of which is incorporated herein by reference in its entirety). As a result, the modified MAb^{P} did not contain glycans with the plant specific α(1,3)-linked Fuc residues. This in turn minimized the risk of immunogenic and allergenic reactions to this epitope in humans.

The α(1,3)-linked Fuc residue is recognized by both IgG and IgE (Wilson et al. (1998) Glycobiology 8:651-661, incorporated herein by reference in its entirety). If present, the xylose residue that is α(1,2)-linked to the β-linked core mannose of the sugars attached to MAb^{P} forms part of the anti-α(1,3)-linked Fuc antibody epitope, but does not on its own constitute a potent epitope. Moreover, the xylose-containing glycans in MAb^{P} are also known to contain an α1,3-antenna and, on these grounds too, the xylose is unlikely to bind IgE. In contrast, α-Gal residues are known to be potent antigens. The terminal α-Gal residues on the sugars attached to MAb^{M} are likely to be accessible even in the context of the IgG CH2 domains in which the glycans are sequestered. Interestingly, the detection of the α-Gal residue provides evidence that the hybridoma cell line had used the murine rather than the human glycosylation machinery.

Rabies MAb^{P} of the invention had similar in vitro neutralizing activity against cell culture adapted virus strains and a street virus, compared to the MAb^{M}. Neutralization depends on blocking of binding sites on the virion and may be mostly mediated by steric hindrance resulting from the relatively large size of the antibody molecule. The altered glycosylation on the CH2 domain of antibodies does not affect their affinity for antigen (Rudd et al. (2001) Science 291:2370-2376; and Wright et al., (1997) Trends. Biotechnol. 15:26-32 each of which is incorporated herein by reference in its entirety).

Rabies MAb^{P} with oligomannose type N-glycans was rapidly cleared in vivo compared to MAb^{M}. The shorter half-life of the MAb^{P} containing oligomannose type glycans did not adversely affect the immunological protection against rabies for post-exposure prophylaxis. The dual effect of rabies post-exposure treatment with both antibody and vaccine in mammals occasionally leads to interference between passive and active immunization because of larger persistence of the antibody in the circulation (Koprowski et al. (1952) J.Immunol. 72:79-84; Schumacher et al. (1992) Vaccine 10:754-760; and Lang et al. (1998) Bull. World Health Org. 76:491495, each of which is incorporated herein by reference in its entirety). Thus, the shorter half-life of this MAb^{P} of the invention offers certain advantage to the current commercial antibody-vaccine prophylaxis since there will be less probability of interference between the passive and active immunity.

Rabies MAb^{P} of the invention was expressed and fully assembled in plants without any gene silencing. The concentration of rabies MAb^{P} in plant was in the range of about 0.01% to 5%, for example, 0.07%, 0.1%, 1%, 2.5%, or 5% of the total soluble protein in plants. It is intended herein that by recitation of such specified ranges, the ranges recited also include all those specific integer amounts between the recited ranges. For example, in the range about 0.1 to 1%, it is intended to also encompass 0.2, 0.3, 0.4, 0.5, 0.6, etc.

2. Plant Expression Vectors

Also encompassed within the scope of the invention are plant expression vectors containing the gene constructs of the invention. Expression vectors are defined herein as DNA sequences that are required for the transcription of cloned copies of genes and the translation of their mRNAs in an appropriate host. Such expression vectors are used to express eukaryotic and prokaryotic genes in plants. Expression vectors include, but are not limited to, cloning vectors, modified cloning vectors, specifically, designed plasmids or viruses.

According to one embodiment of the invention, there are provided plant expression vectors containing one or more gene constructs of the invention carrying the antibody genes, including antibody subunit genes or fragments thereof. The plant expression vectors of the invention contain the necessary elements to accomplish genetic transformation of plants so that the gene constructs are introduced into the plant's genetic material in a stable manner, i.e., a manner that will allow the antibody genes to be passed on to the plant's progeny. The design and construction of the expression vector influence the integration of the gene constructs into the plant genome and the ability of the antibody genes to be expressed by plant cells.

Preferred among expression vectors are vectors carrying a functionally complete human or mammalian heavy or light chain sequence having appropriate restriction sites engineered so that any variable V_{H} or variable V_{L} chain sequence with appropriate cohesive ends can be easily inserted therein. Human C_{H} or C_{L} chain sequence-containing vectors are thus an embodiment of the invention and can be used as intermediates for the expression of any desired complete H or L chain in any appropriate host.

Many vector systems are available for the expression of cloned HC and LC genes in host cells. Different approaches can be followed to obtain complete HC and LC subunit antibodies. In one embodiment, HC and LC were co-expressed in the same cells to achieve intracellular association and linkage of HC and LC into complete tetrameric HC and LC antibodies. The co-expression can occur by using either the same or different plasmids in the same host.

Polynucleotides encoding both HC and LC are placed under the control of one or more different or the same promoters, for example in the form of a dicistronic operon, into the same or different expression vectors. The expression vectors are then transformed into cells, thereby selecting directly for cells that express both chains.

In one embodiment, the polynucleotide encoding LC and polynucleotides encoding HC are present on two mutually compatible expression vectors which are each under the control of different or the same promoter(s). In this embodiment, the expression vectors are co-transformed or transformed individually. For example, cells are transformed first with an expression vector encoding one chain, for example LC, followed by transformation of the resulting cell with an expression vector encoding a HC.

In a preferred embodiment, a single expression vector carrying polynucleotides encoding both the HC and LC is used. Cell lines expressing HC and LC molecules are transformed with expression vectors encoding additional copies of LC, HC, or LC plus HC in conjunction with additional selectable markers to generate cell lines with enhanced properties, such as higher production of assembled HC and LC antibody molecules or enhanced stability of the transformed cell lines.

Specifically designed expression vectors allow the shuttling of DNA between hosts, such as between bacteria and plant cells. According to a preferred embodiment of the invention, the expression vector contains an origin of replication for autonomous replication in host cells, selectable markers, a limited number of useful restriction enzyme sites, active promoter(s), and additional regulatory control sequences.

Preferred among expression vectors, in certain embodiments, are those expression vectors that contain cis-acting control regions effective for expression in a host operatively linked to the polynucleotide of the invention to be expressed. Appropriate trans-acting factors are supplied by the host, supplied by a complementing vector or supplied by the vector itself upon introduction into the host.

In certain preferred embodiments in this regard, the expression vectors provide for specific expression. Such specific expression is an inducible expression, cell or organ specific expression, host-specific expression, or a combination thereof.

In a preferred embodiment of the invention, the plant expression vector is an Agrobacterium-based expression vector. Various methods are known in the art to accomplish the genetic transformation of plants and plant tissues by the use of Agrobacterium-mediated transformation systems, i.e., A. tumefaciens and A. rhizogenesis. Agrobacterium is the etiologic agent of crown gall, a disease of a wide range of dicotyledons and gymnosperms that results in the formation of tumors or galls in plant tissue at the site of infection. Agrobacterium, which normally infects the plant at wound sites, carries a large extrachromosomal element called Ti (tumor-inducing) plasmid.

Ti plasmids contain two regions required for tumor induction. One region is the T-DNA (transferred-DNA) which is the DNA sequence that is ultimately found stably transferred to plant genomic DNA. The other region is the vir (virulence) region which has been implicated in the transfer mechanism. Although the vir region is absolutely required for stable transformation, the vir DNA is not actually transferred to the infected plant. Transformation of plant cells mediated by infection with A. tumefaciens and subsequent transfer of the T-DNA alone have been well documented. See, i.e., Bevan et al. (1982) Int. Rev. Genet. 16:357, incorporated herein by reference in its entirety.

A. rhizogenes has also been used as a vector for plant transformation. This bacterium, which incites root hair formation in many dicotyledonous plant species, carries a large extrachromosomal element called a Ri (root-inducing) plasmid which functions in a manner analogous to the Ti plasmid of A. tumefaciens. Transformation using A. rhizogenes has developed analogously to that of A. tumefaciens and has been successfully utilized to transform the plant of this invention.

Agrobacterium system has been developed to permit routine transformation of a variety of plant tissues. Representative tissues transformed by this technique include, but are not limited to, tobacco, tomato, sunflower, cotton, rapeseed, potato, poplar, and soybean, among others.

2.1. Promoters

Promoters are responsible for the regulation of the transcription of DNA into mRNA. A number of promoters which function in plant cells are known in the art, and may be employed in the practice of the present invention. These promoters are obtained from a variety of sources such as, for example, plants or plant viruses, bacteria, among others.

The invention, as described and disclosed herein, encompasses the use of constitutive promoters, inducible promoters, or both.

In general, an “inducible promoter” is a promoter that is capable of directly or indirectly activating transcription of one or more DNA sequences or genes in response to an inducer. In the absence of an inducer the DNA sequences or genes will not be transcribed. Typically the protein factor, that binds specifically to an inducible promoter to activate transcription, is present in an inactive form which is then directly or indirectly converted to the active form by the inducer. The inducer can be a chemical agent such as a protein, metabolite, growth regulator, herbicide or phenolic compound or a physiological stress imposed directly by heat, cold, wound, salt, or toxic elements, light, desiccation, pathogen infection, or pest-infestation.

Inducible promoters are determined using any methods known in the art. For example, the promoter may be operably associated with an assayable marker gene such as GUS (glucouronidase), the host plant can be engineered with the construct; and the ability and activity of the promoter to drive the expression of the marker gene in the harvested tissue under various conditions assayed.

A plant cell containing an inducible promoter is exposed to an inducer by externally applying the inducer to the cell or plant such as by spraying, harvesting, watering, heating or similar methods. In addition, inducible promoters include tissue specific promoters that function in a tissue specific manner to regulate the gene of interest within selected tissues of the plant Examples of such tissue specific promoters include seed, flower or root specific promoters as are well known in the field.

A “constitutive promoter” is a promoter that directs the expression of a gene throughout the various parts of a plant and continuously throughout plant development.

In one embodiment of the invention, promoters are tissue-specific. Non-tissue-specific promoters (i.e., those that express in all tissues after induction), however, are preferred. More preferred are promoters that additionally have no or very low activity in the uninduced state. Most preferred are promoters that additionally have very high activity after induction. Particularly preferred among inducible promoters are those that can be induced to express a protein by environmental factors that are easy to manipulate.

In a preferred embodiment of the invention, one or more constitutive promoters are used to regulate expression of antibody genes or antibody subunit genes in a plant.

Examples of an inducible and/or constitutive promoters include, but are not limited to, promoters isolated from the caulimovirus group such as the cauliflower mosaic virus 35S promoter (CaMV35S), the enhanced cauliflower mosaic virus 35S promoter (enh CaMV35S), the figwort mosaic virus full-length transcript promoter (FMV35S), the promoter isolated from the chlorophyll a/b binding protein, proteinase inhibitors (PI-I, PI-II), defense response genes, phytoalexin biosynthesis, phenylpropanoid phytoalexin, phenylalanine ammonia lyase (PAL), 4-coumarate CoA ligase (4CL), chalcone synthase (CHS), chalcone isomerase (CHI), resveratrol (stilbene) synthase, isoflavone reductase (IFR), terpenoid phytoalexins , HMG-CoA reductase (HMG), casbene synthetase, cell wall components, lignin, phenylalanine ammonia lyase, cinnamyl alcohol dehydrogenase (CAD), caffeic acid o-methyltransferase, lignin-forming peroxidase, hydroxyproline-rich glycoproteins (HRGP), glycine-rich proteins (GRP), thionins, hydrolases, lytic enzymes, chitinases (PR-P, PR-Q), class I chitinase, basic, Class I and II chitinase, acidic, class II chitinase, bifunctional lysozyme, β-1,3-Glucanase, arabidopsis, β-fructosidase, superoxide dismutase (SOD), lipoxygenase, prot., PR1 family, PR2, PR3, osmotin, PR5, ubiquitin, wound-inducible genes, win1, win2 (hevein-like), wun1, wun2, nos, nopaline synthase, ACC synthase, HMG-CoA reductase hmgl, 3-deoxy-D-arabino-heptulosonate-7-phosphate synthase, HSP7033, Salicylic acid inducible acid peroxidase, PR-proteins, glycine-rich protein, methyl jasmonate inducible, vspB⁴², heat-shock genes, HSP70, cold-stress inducible, drought, salt stress, hormone inducible, gibberellin, α-amylase, abscisic acid, EM-1, RAB, LEA genes, ethylene, phytoalexin biosyn.genes, or a combination thereof.

The above-noted promoters are listed solely by way of illustration of the many commercially available and well known plant promoters that are available to those of skill in the art for use in accordance with this aspect of the present invention. It will be appreciated that any other plant promoter suitable for, for example, introduction, maintenance, propagation or expression of a polynucleotide or polypeptide of the invention in plants may be used in this aspect of the invention.

2.3. Regulatory Control Elements

Gene constructs of the present invention can also include other optional regulatory elements that regulate, as well as engender, expression. Generally such regulatory control elements operate by controlling transcription. Examples of such regulatory control elements include, for example, enhancers (either translational or transcriptional enhancers as may be required), repressor binding sites, terminators, leader sequences, and the like.

Specific examples of these elements include, but are not limited to, the enhancer region of the 35S regulatory region, as well as other enhancers obtained from other regulatory regions, and/or the ATG initiation codon and adjacent sequences. The initiation codon must be in phase with the reading frame of the coding sequence to ensure translation of the entire sequence. The translation control signals and initiation codons are from a variety of origins, both natural and synthetic. Translational initiation regions are provided from the source of the transcriptional initiation region, or from the structural gene. The sequence is also derived from the promoter selected to express the gene, and can be specifically modified to increase translation of the mRNA.

The nontranslated leader sequence is derived from any suitable source and is specifically modified to increase the translation of the mRNA. In one embodiment, the 5′ nontranslated region is obtained from the promoter selected to express the gene, the native leader sequence of the gene, coding region to be expressed, viral RNAs, suitable eucaryotic genes, or a synthetic gene sequence, among others.

In another embodiment, gene constructs of the present invention comprise a 3U untranslated region. A 3U untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3U end of the mRNA precursor.

The termination region or 3′ nontranslated region is employed to cause the termination of transcription and the addition of polyadenylated ribonucleotides to the 3′ end of the transcribed mRNA sequence. The termination region may be native with the promoter region, native with the structural gene, or may be derived from the expression vector or another source, and would preferably include a terminator and a sequence coding for polyadenylation. Suitable 3′ nontranslated regions of the chimeric plant gene include, but are not limited to: (1) the 3′ transcribed, nontranslated regions containing the polyadenylation signal of Agrobacterium tumor-inducing (Ti) plasmid genes, such as the nopaline synthase (NOS) gene, and (2) plant genes like the soybean 7S storage protein genes and the pea small subunit of the ribulose 1,5-bisphosphate carboxylase-oxygenase, among others.

The addition of appropriate introns and/or modifications of coding sequences for increased translation can also substantially improve foreign gene expression. Appropriate introns include, but are not limited to, the maize hsp70 intron, maize adh 1 intron, and rice actin intron.

In a preferred embodiment, the regulatory control elements of the invention include an alfalfa mosaic virus untranslated leader sequence and Lys-Asp-Glu-Leu (KDEL) endoplasmic reticulum retention signal operably attached to the N- and C-terminus of heavy chain, respectively.

It has been shown that the inclusion of KDEL or HDEL amino acid sequences at the carboxy terminus of at least one protein enhanced the recognition for that protein by the plant endoplasmic reticulum retention machinery. See, Munro and Pelham (1987) Cell 48:988-997; Denecke et al. (1991) EMBO-J: 11:2345; Herman et al. (1991) Planta 182:305; and Wandelt et al. (1992) The Plant Journal 2:181, each of which is incorporated herein by reference in its entirety.

2.4. Selectable Markers

To aid in identification of transformed plant cells, the gene constructs of this invention may be further manipulated to include selectable marker genes that are functional in bacteria, plants or both. Useful selectable markers include, but are not limited to, enzymes which provide for resistance to an antibiotic such as ampicillin resistance gene (Amp^{r}), tetracycline resistance gene (Tc^{r}), cycloheximide-resistance L41 gene, the gene conferring resistance to antibiotic G418 such as the APT gene derived from a bacterial transposon Tn903, the antibiotic hygromycin B-resistance gene, gentamycin resistance gene, and/or kanamycine resistance gene, among others. Similarly, enzymes providing for production of a compound identifiable by color change such as GUS, or luminescence, such as luciferase, are possible.

A selectable marker gene is used to select transgenic plant cells of the invention, which transgenic cells have integrated therein one or more copies of the gene construct of the invention. The selectable or screenable genes provides another check for the successful culturing of cells carrying the genes of interest. Transformed plant calli may be selected by growing the cells on a medium containing, for example, kanamycin.

3. Transformation Strategies

Host plants are genetically transformed to incorporate one or more gene constructs of the invention. There are numerous factors which influence the success of plant transformation. The design and construction of the expression vector influence the integration of the foreign genes into the genome of the host plant and the ability of the foreign genes to be expressed by plant cells. The type of cell into which the gene construct is introduced must, if whole plants are to be recovered, be of a type which is amenable to regeneration, given an appropriate regeneration protocol.

The integration of the polynucleotides encoding the desired gene into the plant host is achieved through strategies that involve, for example, insertion or replacement methods. These methods involve strategies utilizing, for example, direct terminal repeats, inverted terminal repeats, double expression cassette knock-in, specific gene knock-in, specific gene knock-out, random chemical mutagenesis, random mutagenesis via transposon, and the like. The expression vector is, for example, flanked with homologous sequences of any non-essential plant genes, bacteria genes, transposon sequence, or ribosomal genes. Preferably the flanking sequences are T-DNA terminal repeat sequences. The DNA is then integrated in host by homologous recombination occurred in the flanking sequences using standard techniques.

In a preferred embodiment of the invention, Agrobacterium-based transformation strategy is employed to introduce the gene constructs into plants. Such transformations preferably use binary Agrobacterium T-DNA vectors (Bevan (1984) supra), and the co-cultivation procedure (Horsch et al. (1985) Science 227:1229-1231, incorporated herein by reference in its entirety). Generally, the Agrobacterium transformation system is used to engineer dicotyledonous plants. The Agrobacterium transformation system may also be used to transform as well as transfer DNA to monocotyledonous plants and plant cells. See, for example, Hernalsteen et al. (1984) EMBO J. 3:3039-3041; Hooykass-Van Slogteren et al. (1984) Nature 311:763-764; Grimsley et al. (1987) Nature 325:1677-179; Boulton et al. (1989) Plant Mol. Biol. 12:3140.; Gould et al. (1991) Plant Physiol. 95:426-434, each of which is incorporated herein by reference in its entirety.

In other embodiments, various alternative methods for introducing recombinant nucleic acid constructs into plants and plant cells are also utilized. These other methods are particularly useful where the target is a monocotyledonous plant or plant cell. Alternative gene transfer and transformation methods include, but are not limited to, protoplast transformation through calcium-polyethylene glycol (PEG)- or electroporation-mediated uptake of naked DNA. See, for example, Paszkowski et al. (1984) EMBO J. 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276, each of which is incorporated herein by reference in its entirety. Electroporation of plant tissues are also disclosed in D'Halluin et al. (1992) Plant Cell 4:1495-1505, incorporated herein by reference in its entirety. Additional methods for plant cell transformation include microinjection, silicon carbide mediated DNA uptake (see, for example, Kaeppler et al. (1990) Plant Cell Reporter 9:415418), and microprojectile bombardment (see, for example, Klein et al. (1988) Proc. Nat. Acad. Sci. USA 85:4305-4309; Gordon-Kamm et al. (1990) Plant Cell 2:603-618, each of which is incorporated herein by reference in its entirety.

In the case of direct gene transfer, the gene construct is transformed into plant tissue without the use of the Agrobacterium plasmids. Direct transformation involves the uptake of exogenous genetic material into plant cells or protoplasts. Such uptake may be enhanced by use of chemical agents or electric fields. The exogenous material may then be integrated into the nuclear genome. The early work with direct transfer was conducted in the Nicotiana tobacum (tobacco) where it was shown that the foreign DNA was incorporated and transmitted to progeny plants. Several monocot protoplasts have also been transformed by this procedure including maize and rice.

Liposome fusion has also been shown to be a method for transforming plant cells. Protoplasts are brought together with liposomes carrying the desired gene. As membranes merge, the foreign gene is transferred to the protoplasts.

Alternatively, exogenous DNA can be introduced into cells or protoplasts by microinjection. In this technique, a solution of the plasmid DNA or DNA fragment is injected directly into the cell with a finely pulled glass needle.

A more recently developed procedure for direct gene transfer involves bombardment of cells by micro-projectiles carrying DNA. In this procedure, commonly called particle bombardment, tungsten or gold particles coated with the exogenous DNA are accelerated toward the target cells. The particles penetrate the cells carrying with them the coated DNA. Microparticle acceleration has been successfully demonstrated to lead to both transient expression and stable expression in cells suspended in cultures, protoplasts, immature embryos of plants including but not limited to onion, maize, soybean, and tobacco.

In addition to the methods described above, a large number of methods are known in the art for transferring cloned DNA into a wide variety of plant species, including gymnosperms, angiosperms, monocots and dicots. Minor variations make these technologies applicable to a broad range of plant species.

4. Transgenic Plants

The invention further relates to transgenic plants, including whole plants, plant organs (i.e., leaves, stems, flowers, roots, etc.), seeds and plant cells (including tissue culture cells), and progeny of same that are transformed with a gene construct according to the invention.

Once plant cells have been transformed, there are a variety of methods for regenerating plants. The particular method of regeneration will depend on the starting plant tissue and the particular plant species to be regenerated. In general, transformed plant cells are cultured in an appropriate medium, which contain selective agents such as, for example, antibiotics, where selectable markers are used to facilitate identification of transformed plant cells. Once callus forms, embryo or shoot formation are encouraged by employing the appropriate plant hormones in accordance with known methods and the shoots transferred to rooting medium for regeneration of plants. The plants are then used to establish repetitive generations, either from seeds or using vegetative propagation techniques. The presence of a desired gene, or gene product, in the transformed plant may be determined by any suitable method known to those skilled in the art. Included in these methods are southern, northern, and western blot techniques, ELISA, and bioassays.

In recent years, it has become possible to regenerate many species of plants from callus tissue derived from plant explants. The plants which can be regenerated from callus include monocots, such as, but not limited to, corn, rice, barley, wheat, and rye, and dicots, such as, but not limited to, sunflower, soybean, cotton, rapeseed and tobacco.

5. Plant-Derived Antibodies and Fragnients Thereof

The invention provides plant-derived human, humanized or chimeric antibodies, including antibody subunits and fragments thereof, with specificity to viral antigens such as rabies antigens. The antibodies of the invention include antibodies that are expressed and isolated by recombinant means from a transgenic plant.

In one embodiment, the antibodies include immunoglobulin molecules having H and L chains associated so that the overall molecule exhibits the desired antigen binding and recognition properties. Various types of immunoglobulin molecules are provided: monovalent, divalent, multivalent, or molecules with the specificity-determining V binding domains attached to moieties carrying desired functions.

In another embodiment, the invention provides for fragments of chimeric immunoglobulin molecules such as Fab, Fab′, or F(ab′)₂ molecules or those proteins coded by truncated genes to yield molecular species functionally resembling these fragments. A chimeric immunoglobulin molecule comprises a chain containing a constant (C) region substantially similar to that present in a natural human immunoglobulin, and a variable (V) region having the desired anti-tumor or antiviral specificity of the invention. Antibodies having chimeric H chains and L chains of the same or different V region binding specificity are prepared by appropriate association of the desired polypeptide chains.

The immunoglobulin molecules are encoded by genes which include the kappa, lambda, alpha, gamma, delta, epsilon or mu constant regions, as well as any number of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains. The human IgG heavy chains are further sub-classified based on their sequence variation, and the subclasses are designated IgG1, IgG2, IgG3 and IgG4.

Antibodies comprise two pairs of a light and heavy domains. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), and framework region 4 (FR4) which constitute the antibody-antigen recognition domain.

In general, as used herein, the term plant-derived antibody or plant-derived monoclonal antibody (MAb^{P}) encompasses a variety of modifications, particularly those that are present in polypeptides expressed by polynucleotides in a host cell. It will be appreciated that polypeptides often contain amino acids other than the 20 amino acids commonly referred to as the naturally occurring amino acids, and that many amino acids, including the terminal amino acids, may be modified in a given polypeptide, either by natural processes, such as processing and other post-translational modifications, or by chemical modification techniques.

Modifications occur anywhere in a polypeptide, including the peptide backbone, the amino acid side chains and the amino or carboxyl termini. Blockage of the amino or carboxyl group in a polypeptide, or both, by a covalent modification, occur in natural or synthetic polypeptides. Such modifications may be present in the antibody polypeptides of the present invention, as well. In general, the nature and extent of the modifications are determined by the host cell's post-translational modification capacity and the modification signals present in the polypeptide amino acid sequence. It will be appreciated that the same type of modification may be present in the same or varying degrees at several sites in a polypeptide.

The plant-derived antibody according to the invention includes truncated and/or N-terminally or C-terminally extended forms of the antibody, analogs having amino acid substitutions, additions and/or deletions, allelic variants and derivatives of the antibody, so long as their sequences are substantially homologous to the native human or mammalian-derived antibody and have specificity to a virus and in particular rabies virus. Other viruses encompassed within the scope of the invention are enveloped viruses, non-enveloped viruses, DNA viruses, RNA viruses, among others. In general, the virus can be from a family of virus including, for example, picornaviridae, calciviridae, stroviride, togaviridae, flavivirdae, coronaviridae, rhabdoviridae (particularly lyssavirus such as rabies virus) filoviridae, paramyxoviridae, orthomyxoviridae, bunyaviridae, arenaviridae, reoviridae, retroviridae, papoviridae, adenoviridae, parvoviridae, herpesviridae, poxviridae, hapadnaviridae, among others.

Variations in the structure of plant-derived antibodies may arise naturally as allelic variations, as disclosed above, due to genetic polymorphism, for example, or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules.

Substitutions may be designed based on, for example, the model of Dayhoff et al. (1978) Atlas of Protein Sequence and Structure, Natl. Biomed. Res. Found. Washington, D.C. These modifications can result in changes in the amino acid sequence, provide silent mutations, modify a restriction site, or provide other specific mutations.

The conserved and variable sequence regions of a plant-derived antibody and the homology thereof can be determined by techniques known to the skilled artisan, such as sequence alignment techniques. For example, the determination of percent identity between two sequences can also be accomplished using a mathematical algorithm, as described above.

6. Polynucleolides Encoding Antibody Polypeptides

This invention also encompasses polynucleotides that correspond to and code for the antibody polypeptides. Nucleic acid sequences are either synthesized using automated systems well known in the art, or derived from a gene bank.

It will be appreciated that a great variety of modifications have been made to DNA and RNA that serve many useful purposes known to those of skill in the art. The polynucleotides of the invention embrace chemically, enzymatically or metabolically modified forms of polynucleotides.

The polynucleotides of the present invention encode, for example, the coding sequence for the structural gene (i.e., antibody gene), and additional coding or non-coding sequences. Examples of additional coding sequences include, but are not limited to, sequences encoding a secretory sequence, such as a pre-, pro-, or prepro- protein sequences. Examples of additional non-coding sequences include, but are not limited to, introns and non-coding 5′ and 3′ sequences, such as the transcribed, non-translated sequences that play a role in transcription and mRNA processing, including splicing and polyadenylation signals, for example, for ribosome binding and stability of mRNA.

The polynucleotides of the invention also encode a polypeptide which is the mature protein plus additional amino or carboxyl-terminal amino acids, or amino acids interior to the mature polypeptide (when the mature form has more than one polypeptide chain, for instance). Such sequences play a role in, for example, processing of a protein from precursor to a mature form, may facilitating protein trafficking, prolonging or shortening protein half-life or facilitating manipulation of a protein for assay or production, among others. The additional amino acids may be processed away from the mature protein by cellular enzymes.

In sum, the polynucleotides of the present invention encodes, for example, a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

The polynucleotides of the invention include “variant(s)” of polynucleotides, or polypeptides as the term is used herein. Variants include polynucleotides that differ in nucleotide sequence from another reference polynucleotide. Generally, differences are limited so that the nucleotide sequences of the reference and the variant are closely similar overall and, in many regions, identical. As noted below, changes in the nucleotide sequence of the variant my be silent. That is, they may not alter the amino acids encoded by the polynucleotide. Where alterations are limited to silent changes of this type, a variant will encode a polypeptide with the same amino acid sequence as the reference.

Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence. According to a preferred embodiment of the invention, there are no alterations in the amino acid sequence of the polypeptide encoded by the polynucleotides of the invention, as compared with the amino acid sequence of the wild type or marnmalian derived peptide.

The present invention further relates to polynucleotides that hybridize to the herein described sequences. The term “hybridization under stringent conditions” according to the present invention is used as described by Sambrook et al. (1989) Molecular Cloning, A Laboratory Manual, Cold Spring Harbor Laboratory Press 1.101-1.104. Preferably, a stringent hybridization according to the present invention is given when after washing for an hour with 1% SSC and 0.1% SDC at 50° C., preferably at 55° C., more preferably at 62° C., most preferably at 68° C. a positive hybridization signal is still observed. A polynucleotide sequence which hybridizes under such washing conditions with the nucleotide sequence shown in any sequence disclosed herein or with a nucleotide sequence corresponding thereto within the degeneration of the genetic code is a nucleotide sequence according to the invention.

The polynucleotides of the invention include polynucleotide sequences that have at least about 50%, 65%, 70%, 75%, 80%, 85%, 90%, 95%, 98%, 99% or more nucleotide sequence identity to the polynucleotides or a transcriptionally active fragment thereof. To determine the percent identity of two amino acid sequences or two nucleic acid sequences, the sequences are aligned for optimal comparison purposes (i.e., gaps can be introduced in the sequence of a first amino acid or nucleic acid sequence for optimal alignment with a second nucleic acid sequence). The amino acid residue or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST program of Altschul et al. (1990), J. Mol. Biol. 215:403-410. BLAST nucleotide searches can be performed with the NBLAST program program, score=100, wordlength=12 to obtain nucleotide sequences homologous to a nucleic acid molecules of the invention. The BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al. (1997) Nucleic Acids Res. 25:3389-3402.

Alternatively, PSI-Blast can be used to perform an iterated search which detects distant relationships between molecules (Id.). When utilizing BLAST, Gapped BLAST and PSI-Blast programs, the default parameters of the respective programs (i.e., XBLAST and NBLAST program can be used (see, HTrP://WWW.NCBI.NLM.NIH.GOV). Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0) which is part of the GCG sequence aliginent software package. When utilizing the ALIGN pfogram for comparing amino acid sequences of a PAM 120 weight residue table, a gap length penalty of 12 and a gap penalty of 4 can be used. In an alternate embodiment, alignments can be obtained using the NA-MULTIPLE-ALIGNMENT 1.0 program, using a GapWeight of 5 and a GapLengthWeight of 1.

7. Methods of Using Plant-Derived Andbodies

In one aspect the invention as described herein provides methods for using the plant-derived antibodies. The plant-derived antibodies of the invention are used for therapeutic and/or diagnostic purposes by themselves, for example, acting via complement-mediated lysis and antibody-dependent cellular cytotoxicity, or coupled to other therapeutic moieties, such as ricin, radionuclides, drugs, etc. The antibodies may be advantageously utilized in combination with factors, such as lymphokines, colony-stimulating factors, and the like, which increase the number or activity of antibody-dependent effector cells.

In one embodiment, the plant-derived antibody of the invention, preferably having human C region, is utilized for passive immunization, especially in humans, with reduced negative immune reactions such as serum sickness or anaphylactic shock, as compared to the mammalian- derived counterpart antibodies.

In another embodiment, the plant-derived antibody of the invention is used as an oral vaccine or a DNA vaccine.

In yet another embodiment, the plant-derived antibody of the invention is used in a diagnostic test kit to detect human viral infections or tumor antigens.

8. Test Kits

Also encompassed within the scope of the invention are diagnostic test kits that contain the plant-derived antibody of the invention. The antibodies are utilized in immunodiagnostic assays and kits in detectably labeled form (ie., enzymes, fluorescent labels, etc.), or in immobilized form (on polymeric tubes, beads, etc.) They may also be utilized in labeled form for in vivo imaging, wherein the label can be a radioactive emitter, or a nuclear magnetic resonance contrasting agent such as a heavy metal nucleus, or a X-ray contrasting agent, such as a heavy metal. The antibodies can also be used for in vitro localization of the recognized viral antigen by appropriate labeling.

Detection can be facilitated by coupling the antibody to a detectable agents. Examples of detectable substances include, but are not limited to various enzymes, prosthetic groups, fluorescent materials, luminescent materials, bioluminescent materials, radioactive materials, disperse dyes, and gold particles. Examples of suitable detectable agents, as disclosed above, includes suitable enzymes, i.e., horseradish peroxidase, alkaline phosphatase, betagalactosidase, or acetylcholinesterase; examples of suitable prosthetic group complexes include, but are not limited to streptavidin/biotin and avidin/biotin; examples of suitable fluorescent materials include, but are not limited to umbelliferone, fluorescein, fluorescein isothiocyanate, rhodamine, dichlorotriazinylamine fluorescein, dansyl chloride or phycoerytirin; an example of a luminescent material includes, but is not limited to luminol; examples of bioluminescent materials include, but are not limited to luciferase, luciferin, and aequorin; and examples of suitable radioactive material include, but are not limited to ¹²⁵I, ³⁵S, ¹⁴C, ³H, Tc^{99M}, or Mg⁵².

9. Pharmaceutical Compositions

The present invention also provides pharmaceutical compositions comprising a therapeutically effective amount of one or more plant-derived antibody of the invention or an immunologically active fragment thereof. Administration of the pharmaceutical compositions of the invention, including vaccines, results in a detectable change in the physiology of a recipient subject, preferably by enhancing passive immunity to one or more viral antigens.

A pharmaceutical composition of the invention can confer protection to one or more genotypes of a human virus such as rabies virus. The present invention thus concerns and provides a means for preventing or attenuating infection by at least one viral antigen.

As used herein, a vaccine confers passive immunity and is said to prevent or attenuate a disease if its administration to an individual results either in the total or partial attenuation (i.e.,. suppression) of a symptom or condition of the disease, or in the total or partial immunity of the individual to the disease. The “protection” provided need not be absolute, i.e., the rabies infection need not be totally prevented or eradicated, provided that there is a statistically significant improvement relative to a control population. Protection can be limited to mitigating the severity or rapidity of onset of symptoms of the disease.

The pharmaceutical preparations of the present invention, suitable for inoculation or for parenteral or oral administration, are in the form of sterile aqueous or non-aqueous solutions, suspensions, or emulsions, and can also contain auxiliary agents or excipients that are known in the art. The pharmaceutical composition of the invention can further comprise immunomodulators such as cytolines which accentuate the immune response. (See, i.e., Berkow et al. (1987) eds:. The Merck Manual, Fifteenth Edition, Merck and Co., Rahway, N.J.; Goodman et al. (1990) eds., Goodman and Gilman's The Pharmacological Basis of Therapeutics, Eighth Edition, Pergamon Press, Inc., Elmsford, N.Y.; Avery's Drug Treatment: Principles and Practice of Clinical Pharmacology and Therapeutics, (1987) Third Edition, ADIS Press, LTD., Williams and Wilkins, Baltimore, Md.; and Katzung (1992) ed. Basic and Clinical Pharmacology, Fifth Edition, Appleton and Lange, Norwalk, Conn., which references and references cited therein, are entirely incorporated herein by reference as they show the state of the art.

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system, or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate; talc, sodium chloride, dried skim milk, glycerol, propylene, glycol, water, ethanol, mannitol, sorbitol, trehelose, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The pharmaceutical composition of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Adjuvants can be generally divided into several groups based upon their composition. These groups include lipid micelles, oil adjuvants, mineral salts (for example, AlK(SO₄)₂, AlNa (SO₄)₂, AlNH₄ (SO₄)), silica, kaolin, polynucleotides (for example, poly IC and poly AU nucleic acids), and certain natural substances, for example, wax D from Mycobacterium tuberculosis, substances found in Corynebacterium parvum, or Bordetella pertussis. Preferred adjuvant of the invention includes, for example, Freund's adjuvant (DIFCO), alum adjuvant (Alhydrogel), MF-50 (Chiron) Novasomes™, or micelles, among others.

A composition is said to be “pharmacologically acceptable” if its administration can be tolerated by a recipient patient. Such an agent is said to be administered in a “therapeutically or prophylactically effective amount” if the amount administered is physiologically significant.

The pharmaceutical composition of the invention is administration through various routes, including, oral, subcutaneous, intravenous, intradermal, intramuscular, intraperitoneal, intranasal, transdermal, or buccal routes. Subcutaneous administration is preferred. Parenteral administration are achieved, for example, by bolus injection or by gradual perfusion over time.

A typical regimen for preventing, suppressing, or treating a disease or condition which can be alleviated by a cellular immune response by active specific cellular immunotherapy, comprises administration of an effective amount of the composition as described above, administered as a single treatment, or repeated as enhancing or booster dosages, over a period up to and including one week to about 48 months.

According to the present invention, an “effective amount” of a composition is an amount sufficient to achieve passive immunity against viral antigens. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

This invention is further illustrated by the following examples, which are not to be construed in any way as imposing limitations upon the scope thereof.

### EXAMPLES

### Example 1

### Construction of Plant Transformation Binary Vector

cDNA fragments encoding for MAb S057 light chain (LC, 729 bp) (GenBank access #:AY172960) and heavy chain (HC, 1431 bp) (GenBank access #:AY172957) (Prosniak et al., J. Infectious Dis. In Press (2003)) were arranged into a pBI121 binary vector (Clonetech, Palo Alto, Calif.) as follows. The HC was amplified with primers containing NcoI and XbaI sites (5′-cgccatggactggacctggaggttc-3′(SEQ ID NO: 5) and: 5′-gctctagattagagctcatctttgtgat-ggtgatggtgatgtttacccggggacagggag-3′ (SEQ ID NO: 6), containing the KDEL ER retention signal)) and placed in-frame with the alfalfa mosaic virus untranslated leader sequence (AMV) of RNA4 (Datla et al. (1993) Plant Sci. 94:139-149, incorporated herein by reference) under the control of the cauliflower mosaic virus (CaMV) 35S promoter with duplicated upstream B domains (Ca2p) (Kay et al. (1987) Science 236:1299-1302, incorporated herein by reference) into the pUC9-based vector.

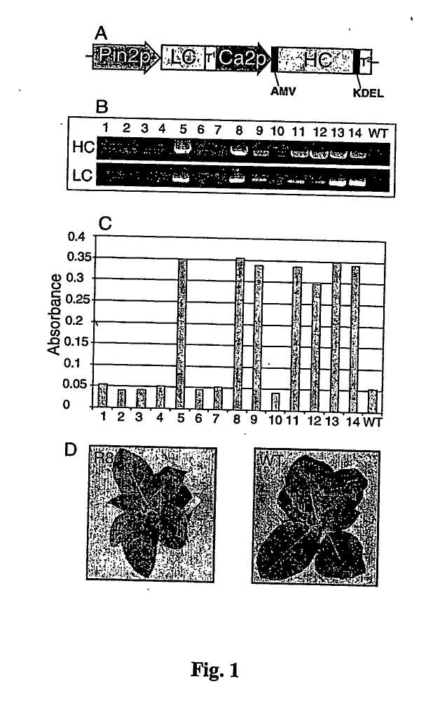

The LC was amplified using primers containing BamHI and PstI sites (5′-cgggatccatgagtgtccccaccatggcc-3′ (SEQ ID NO: 7), and 5′-cgctgcagctatgaacattctgtaggggc-3′ (SEQ ID NO: 8), and subcloned into the corresponding sites of pGEM-T-based vector (Promega, Madison, Wis.) between potato proteinase inhibitor II promoter (Pin2p) and its terminator (Ko et al. (2000) Biotechnol Lett. 22:373-381, incorporated herein by reference). The HC and LC expression cassettes were consequently transferred as HindIII-EcoRI fragment (HC) and HindIII fragment (LC) into the plant binary vector pBI121 to yield pBIRA-57 (FIG. 1A).

FIG. 1A shows the construct arrangement. The HC cDNA was fused with the alfalfa mosaic virus untranslated leader sequence of RNA4 (AMV) at its 5′ end and the KDEL retention signal at its 3′ end and placed downstream of the enhanced Cauliflower Mosaic Virus 35S promoter (Ca2p). The LC was placed under the control of another strong potato proteinase inhibitor II promoter (Pin2p).

### Example 2

### Plant Transformation

Tobacco leaf explants (Nicotiana tabacum cv. Xanthi) were, used for Agrobacterium-mediated transformation (A. tumefaciens EHA105) in MS-based media (Hiatt et al. (1989) Nature 342:76-78) according to the described protocols (Ko et al. (2000) supra). Tobacco transgenic lines were generated by Agrobacterium-mediated plant transformation with a binary vector carrying both the heavy chain (HC) and light chain (LC) of human MAb S057 (FIG. 1). Independent transgenic lines were selected on kanamycin (100 μig/ml). Transgenic tobacco lines were later maintained in soil, and subsequent generations (T₁ and T₂) were obtained by self-fertilization.

### Example 3

### Molecular Characterization of Transgenic Plants

PCR amplification of MAb S057 HC and LC was performed with genomic DNA of each transgenic line using the same primers as described above. PCR analysis of transgenic tobacco plants generated from independent transformation events revealed the presence of both HC and LC in seven transgenic lines (R5, R8, R9, R11, R12, R13 and R14) (FIG. 1B). Protein expression analysis by ELISA confirmed that human MAb S057 is expressed in the R5, R8, R9, R11, R12, R13 and R14 transgenic lines (FIG. 1C). Transgenic line R8 with the highest absorbance level (>0.35) was selected for further studies. Transgenic tobacco plant R8 did not differ morphologically from the wild type (WT) tobacco plant (FIG. 1D) and retained the same level of protein expression over several generations MAb S057 expression levels and assembly.

Transgenic plants were further analyzed by ELISA as follows. 96-well Nunc-Immuno™ MaxiSorp™ Surface plates (Nunc, Denmark) were coated with rabies virus strain CVS-11. Soluble protein was extracted from 0.2 mg of young leaf tissue from transgenic and wild type tobacco plants essentially as described (Ko et al. (1998) J. Amer. Soc. Hort. Sci. 123:11-18, incorporated herein by reference). Plates were loaded with soluble protein leaf extracts and with serial threefold dilutions of 2 μg/ml of MAb S057 purified from the mammalian expression system (MAb^{M}) (Prosniak et al. (2003) J. Infectious. Dis. In Press) used as a positive control. Goat anti-human horseradish peroxidase conjugate (Jackson ImmunoResearch Labs, West Grove, Pa.) was applied and detected using peroxidase-specific substrate O-phenylenediamine dihydrochloride (Sigma, St. Louis, Mo.) according to the manufacturer's recommendations. Absorbance at 490 nm was read on a SPECTRAmax® 340PC Microplate Spectrophotometer (Molecular Devices, Sunnyvale, Calif.).

### Example 4

### SDS-PAGE and Protein Blot Analysis

One gram of tobacco leaf tissues of Example 2 was ground in liquid nitrogen with 100 μl of extraction buffer (50 mM Tris, pH 7.5, 250 mM sucrose) containing protease “complete” inhibitor cocktail (Roche, Indianapolis, Ind.). Forty μg of soluble protein (in 10 μl) was resolved by 12% SDS-PAGE and transferred to Immobilon-P Transfer Membrane (Millipore Corp., Bedford, Ma.) using a mini-Protean II™ system (Bio-Rad Labs, Hercules, Calif.) according to the manufacturer's recommendations. Goat anti-human antibody (Fc_{γ} fragment-specific and F(ab′)₂ fragment-specific) conjugated to horseradish peroxidase (Jackson ImmunoResearch Labs) was applied to detect HC and LC, respectively. The signal was detected using “SuperSignal” chemiluminescence substrate (Pierce, Rockford, Ill.). MAb^{M} was used as a positive control.

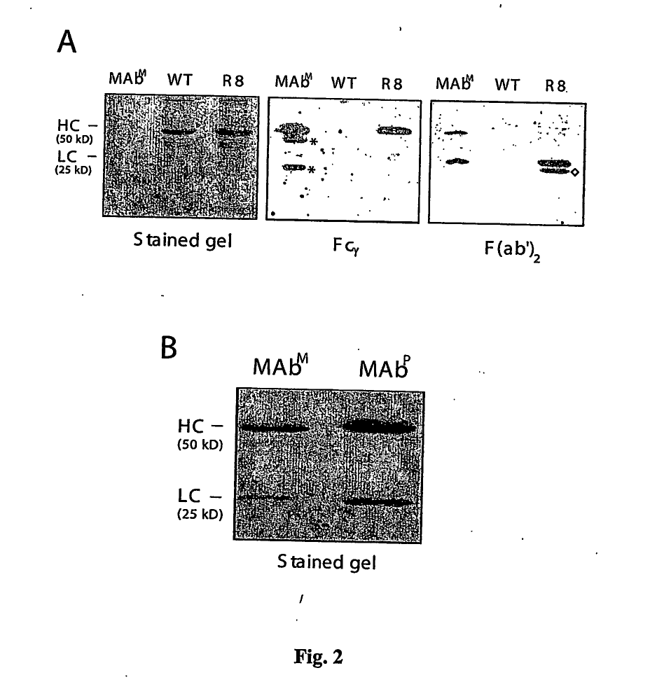

Both HC and LC were identified in the soluble protein extracts from the transgenic plant R8 leaf tissue as major bands migrating at the expected molecular weights, 50 kD and 25 kD, respectively (FIG. 2A). HC was readily detected by anti-human Fc_{γ} fragment specific antibody in both MAb^{M} and R8 (middle panel, FIG. 2A). Two lower molecular weight bands observed in MAb^{M} (FIG. 2A, asterisks) were most likely to be HC proteolytic degradation products. LC was detected by anti-human F(ab′)₂ fragment specific antibody. The HC band was detected in MAb^{M}, whereas only LC was detected in R8 together with a lower molecular weight band probably resulting from proteolytic degradation (right panel, FIG. 2A, diamond).

The expression level of MAb^{P} in R8 plants was calculated as 3 μg/g of fresh leaf weight (0.07% of total soluble protein), consistent with ELISA results. Further purification of antibody derived from plant leaf extracts (MAb^{P}) and from hybridoma supematants (MAb^{M}) using a single- and/or double-step purification with Protein A and Protein G yielded high quality protein products (HC and LC only). FIG. 2B shows the results of the single-step (Protein A) purification.

### Example 5

### Purification of Plant-Derived Antibodies

Soluble protein extracts were subjected to further affinity purification. One-step purification was performed using the batch method immunoprecipitation with 1.5 ml of protein A agarose matrix (Invitrogen, Calif.) for as long as overnight at 4° C. Protein was eluted from the matrix after several washes with protein extraction buffer. For HPLC analysis of N-linked glycans, an extra purification step was carried out on a Protein G column (Pierce) according to the manufacturer's recommendations. The one- or two-step purified protein was dialyzed in 1×PBS buffer and brought to the appropriate concentrations with Millipore spin-columns (10K). Preparations were either used immediately or stored at −80° C. as aliquots.

### Example 6

### In Vitro Rabies Virus Neutralization Assay

The rapid fluorescent focus inhibition test (RFFIT) was carried out with some modifications (Dietzschold et al. (1990) J. Virol. 64:3087-3090; and Champion et al. (2000) J. Immunol. Methods 235:81-90, each of which is incorporated herein by reference in its entirety). Three-fold serial dilutions of MAb^{P}, MAb^{M}, and commercially available HRIG (Enogam Rabies-HT, Aventis Pasteur Inc., Swiftwater, Pa.) were incubated with rabies virus cell culture adapted strains (CVS-1 1 and CVS-N2c) and dog (DRV-4) street rabies virus for 60 min at 37° C. The mixture of antibody and virus was used to infect baby hamster kidney (BHK-21) cells. After incubation in a 96-well flat bottom plate for 40 hr at 37° C., the cells were washed, fixed and stained with the fluorescent isothiocyanate (FITC)-labeled anti-rabies reagent and examined under a fluorescence microscope.

In vitro comparison of the neutralizing activity of MAb^{P}, MAb^{M} and HRIG against cell culture adapted and a street rabies virus (Table 1) indicated that MAb^{P} was as active against the cell culture adapted virus strain CVS-11 as MAb^{M} and HRIG, and more active than HRIG against the CVS-N2c strain. MAb^{P} had stronger activity compared to MAb^{M} and HRIG against the street virus DRV4. Together these data demonstrate that the rabies virus neutralizing activity of MAb^{P} is as high as that of MAb^{M} and/or coniparable to that of HRIG.

TABLE 1
Comparison of virus neutralizing activity of MAb^{P}, MAb^{M},
and HRIG against different rabies viruses.

Results of VNA^{a} (IU/ml)

Antibody

| Rabies viruses used^{b} | MAb^{P} | MAb^{M} | HRIG |
|---|---|---|---|
| CVS-11 | 162 | 162 | 162 |
| CVS-N2c | 108 | 108 | 54 |
| DRV-4 | 81 | 54 | 27 |

^{a}Virus neutralizing antibody (VNA) titer was determined as described (Jobling et al., Nat Biotechnol 21, 77-80 (2003), incorporated herein by reference in its entirety).
^{b}CVS-11 and CVS-N2c are cell culture-adapted virus strains, and DRV-4 is a dog street rabies virus.

### Example 7

### HPLC Analysis of N-Linked Glycans

The HC and LC of MAb^{P} and MAb^{M} proteins were separated on a SDS-PAGE. N-linked glycans were released from gel slices by incubation with PNGase F and PNGase A (Navazio et al. (2002) Biochemistry 41:14141-14149; and Kuster et al. (1997) Anal. Biochem. 250:82-101, each of which is incorporated herein by reference). Labeling and high-performance liquid chromatography (HPLC) and simultaneous exoglycosidase sequencing of the released glycan pool were performed as described (Bigge et al. (1995) Anal. Biochem. 230:229-38; and Rudd et al. (1999) Biotechnol. Genet. Eng. Rev. 16:1-21, each of which is incorporated herein by reference). Exoglycosidases were used at the following concentrations: Arthrobacter ureafaciens sialidase (ABS, EC 3.2.1.18), 1-2 U/ml; almond meal α-fucosidase (AMF, EC 3.2.1.111), 3 mU/ml; bovine testes β-galactosidase (BTG, EC 3.2.1.23), 1-2 U/ml; Streptococcus pneumoniae α-hexosaminidase (SPH, EC 3.2.1.30), 120 U/ml; and Jack Bean α-mannosidase (JBM, EC 3.2.1.24), 100 mU/ml Glycopro® Glucosaminidase (GluH) (Prozyme, San Leandro, Calif.) was used as recommended by the manufacturer.

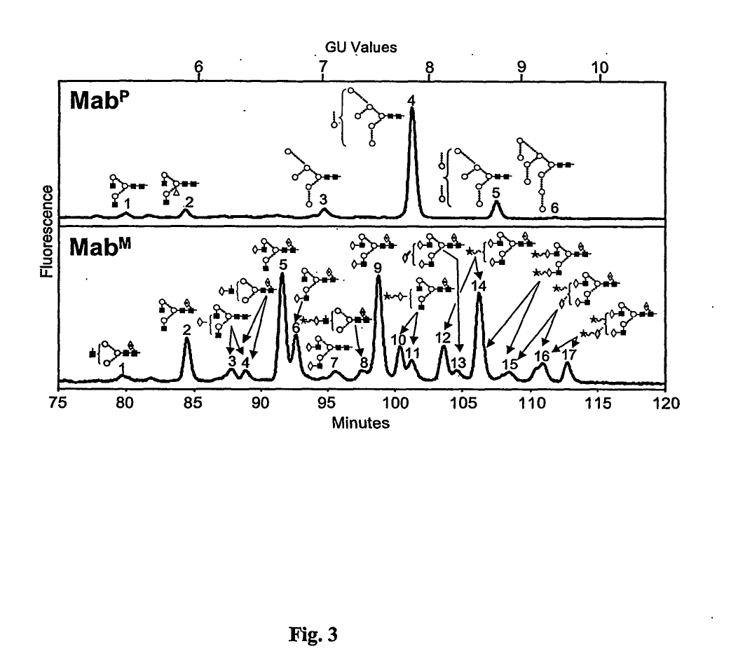

No significant differences were found between the glycan pools obtained by these different enzymatic releases indicating that no α(1,3)-Fuc was present in the MAb^{P}. The HPLC profiles of the fluorescently labeled N-glycan pools, released by PNGase F from MAb^{P} and MAb^{M}, are shown in FIG. 3. Preliminary structures assigned to the glycans in the six peaks in the MAb^{P} profile and 17 peaks in the MAb^{M} profile (FIG. 3) were confirmed by exoglycosidase digestions and were consistent with MALDI-TOF mass spectrometry results.

For MAb^{P} (FIG. 3, top panel), 90% of the total glycan pool consisted of oligomannose type oligosaccharides (peaks 3-6 contained Man₆₋₉GlcNAc₂, respectively). The remaining two peaks (1 and 2) corresponded to GlcNAc₂Man₃GlcNAc₂ (4.3%) and GlcNAc₂Man₃(Xyl)GlcNAc₂ (5.7%). No Fuc or Gal residues were found on any plant-derived glycan structures. In contrast, the MAb^{M} (FIG. 3, bottom panel) displayed a range of complex glycans, most of which (95.4%) contained a core α(1,6)-Fuc and outer-arm Gal and about half contained sialic acid (peaks 7-12 and 14-17). 12% of glycans (within peaks 13, 15-17) contained additional Gal residues. This additional Gal was identified as the terminal monosaccharide in Galoα1-3Galβ1-4GlcNAc-R, since it was digested with coffee bean α-galactosidase that releases only ccGal linked monosacharides.

### Example 8

### Matrix-Assisted Laser Desorption/Ionization

Time-of-flight (MALDI-TOF) mass spectrometry of released glycans. After removing traces of gel (C18 column) prior to analysis underivatized glycans were purified using a Nafion 117 membrane (Aldrich Chemical Co. Ltd., Poole, Dorset, UK) and examined by MALDI mass spectrometer using a positive mode with a Micromass TOFSpec 2E reflectron-TOF instrument (Micromass (UK) Ltd., Wythenshawe, Manchester, U.K.). Samples (0.3 μl in water) were mixed with a saturated solution of 2,5-dihydroxybenzoic acid on the MALDI target and allowed to dry at room temperature. Each sample was then recrystalized from the ethanol.

Operating conditions for the mass spectrometer were: acceleration voltage, 20 kV; pulse voltage, 3200 V; and the delayed extraction ion source was 500 ns. The,instrument was calibrated with dextran oligomers. Monoisotopic masses of the [M+Na]+ ions were within 0.1 mass units of the calculated values. Multiple-stage MALDI fragmentation spectra were acquired on an AXIMA-QIT MALDI quadrupole ion trap time-of-flight instrument controlled by Kratos Launchpad software (Kratos Analytical, Manchester, UK). The nitrogen laser (337 nm, 3 ns pulse width) pulse rate was 10 Hz and a small bias voltage (2.5 to 30 V) was applied to the MALDI sample plate depending on mass of the analyte under investigation. Following ionization, ions were extracted by a negative potential (−10 kV), trapped by application of a retarding potential to the end-cap and an rf potential (500 kHz) to the ring electrode of the ion trap and cooled using helium (6×10⁻³ Torr).

Fragmentation was induced by resonant excitation following application of a supplementary AC potential to the end cap electrodes. Product ions from the molecular or fragment ions were extracted into the TOF analyzer with an accelerating voltage of 10 kV. The TOF was externally calibrated using fullerite deposited directly onto the sample stage. Glycans, subjected to fragmentation, were previously digested with Arthrobacter ureafaciens sialidase (ABS) and the sample target was prepared as above.

Consistent with the HPLC elution position and the exoglycosidase digestions, MALDI mass spectrometry using a tandem ion trap-time-of-flight instrument, as described above, also located the extra galactose to the non-reducing terminus of one of the antennae of the glycan with the composition Hex₆HexNAc₄dHex₁ (Gal₃GlcNAc4Man₃Fuc₁, [M+Na]⁺ at m/z 1971.7). Two successive stages of fragmentation (MS3 spectrum) indicated that the structure of the ion at m/z 550.2, derived from this antenna, was Hex-Hex-HexNAc (data not shown). This Gal residue is therefore the commonly known α-Gal epitope of murine origin that is antigenic to humans (Galili (2001) Biochimie 83:557-563, incorporated herein by reference in its entirety).

### Example 9

### In Vivo Ralf-Life of MABS

Five μg of MAb^{P} or MAb^{M} in 100 μl of 1×PBS buffer were injected intraperitoneally into 10 and 7 BALB/c mice (female, 6-8 weeks) (Jackson Laboratories, Bar Harbor, Me.), respectively. Blood samples were collected from the orbital sinus on days 1, 2, 3, 4, 7, 8, and 10 after injection; each mouse was bled only twice during the entire time period. Serum levels of MAb^{P} and MAb^{M} were detected by ELISA as described above.

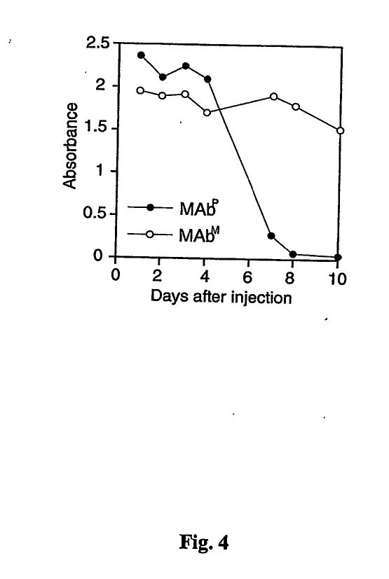

In vivo stability of MAb^{P} and MAb^{M} was analyzed in a comparative clearance test in which mice were injected intraperitoneally and blood samples were collected for up to 10 days. In an ELISA to determine the levels of these antibodies in serum from mice challenged with the CVS-l rabies virus strain, MAb^{P} was barely detectable 10 days after injection, while MAb^{M} was still abundant (FIG. 4).

### Example 10

### In Vivo Protection Assay Against Rabies Virus

Two month-old (100 g) female Syrian hamsters (Harlan, Sprague, Dawley) were inoculated with 50 μl of a homogenate (˜10^{6.8} mouse intracerebral lethal dose (MICLD)₅₀/ml of a Texas coyote rabies virus in circulation at the United States—Mexico border) of salivary gland tissue from a naturally infected rabid coyote as described in Hanlon et al. (2001) Vaccine 19:3834-3842, incorporated herein by reference. The post-exposure prophylaxis protocol was initiated 4 hr after intramuscular inoculation of the rabies virus in the left gastrocnemius muscle. The trial consisted of nine groups (nine hamsters each) and untreated controls. Each treated group intraperitoneally received commercial HIUG (2 IU/animal) (Bayrab®, Bayer Corp., Elkhart, Ind.), and MAb^{P} at 0.4, 0.7, and 3 IU/animal with or without commercial human diploid cell culture rabies virus (HDCV) vaccine (Imovax®, Aventis Pasteur Inc., Swiftwater, Pa.), respectively. The HRIG or MAb^{P} was inoculated in the left gastrocnemius muscle. A tenth group of 3 hamsters received only the HDCV vaccine. The HDCV vaccine was administered in the right muscle at a volume 50 μl undiluted from the vial on days 0, 3, 7, and 14. Animals were observed daily and were sedated with ketamine hydrochloride and then euthanized by CO₂ intoxication on the first day that clinical signs of rabies became evident.

TABLE 2
In vivo efficacy of MAb^{P} for post-exposure Prophylaxis of hamster
injected with a lethal dose of coyote rabies street viruses.
Post-exposure treatment

Vaccine (HDCV)^{b}

| Antibody^{a} (IU/animal) | − | + |
|---|---|---|
| MAb^{P} (3) | 5/9^{c} | 9/9 |
| MAb^{P} (0.7) | 1/9 | 8/9 |
| MAb^{P} (0.4) | 2/9 | 8/9 |
| HRIG (2) | 4/9 | 8/9 |
| Untreated Control | 0/9 | 0/3 |

^{a}IU: International Unit.
^{b}HDCV: commercial (Imovax, lot MO475) human diploid cell culture rabies virus vaccine. “−” and “+”indicate treatments without or with HDCV, respectively.
^{c}Number of surviving hamsters/number of hamsters tested.

Table 2 demonstrates the results of the experiment on the efficacy of MAb^{P} in post-exposure prophylaxis in hamsters injected with rabies virus of coyote origin. In this in vivo protection assay, all untreated control hamsters succumbed to fatal rabies virus encephalitis, as did those animals that received human diploid cell culture rabies virus (HDCV) vaccine only. The survival rate for hamsters that received 3 IU MAb^{P} or 2 IU HIUG without administration of HDCV vaccine was ˜50%, while the rate was decreased in hamsters receiving lower MAb^{P} doses of 0.4 and 0.7 IU alone. The survival rate remained high (8 out of 9) when MAb^{P} at any concentration level was administered together with HDCV vaccine (Table 2).

The present invention may be embodied in other specific methods, products, and forms without departing from its spirit of essential characteristics. The embodiments and examples provided in this specification are intended to illustrate the principles of the invention, but not to limit its scope. Various other embodiments, examples, modifications, and equivalents to the embodiments and examples provided in this specification may occur to those skilled in the art upon reading the present disclosure or practicing the present invention. Such variations, modifications, examples, and equivalents are intended to come within the scope of the invention. The contents of all references, patents and published patent applications cited throughout this application are expressly incorporated herein by reference.

What is claimed is:

1. A plant-derived human monoclonal antibody comprising a rabies MAb^{P}, wherein the rabies MAb^{P} contains predominantly oligomannose type N-glycans and has substantially reduced or no α(1,3)-linked fucose residues.

2. The plant-derived human monoclonal antibody of claim 1, wherein the rabies MAb^{P} is derived from rabies human MAb S057.

3. The plant-derived human monoclonal antibody of claim 1, wherein the rabies MAb^{P} is encoded by a polynucleotide molecule comprising SEQ ID NO: 1, SEQ ID NO: 3, or a combination thereof, or a pol.ynucleotide molecule having a sequence that is substantially homologous to SEQ ID NO: 1, SEQ ID NO: 3, or a combination thereof.

4. The plant-derived human monoclonal antibody of claim 1, wherein the rabies MAb^{P} comprises a polypeptide molecule comprising SEQ ID NO: 2, SEQ ID NO: 4, or a combination thereof, or a polypeptide molecule having a sequence that is substantially homologous to SEQ ID NO: 2, SEQ ID NO: 4, or a combination thereof.

5. An expression vector comprising, one or more gene constructs comprising polynucleotides encoding one or more rabies human MAb S057 subunits under the control of one or more promoters, operatively linked to regulatory control elements and Agrobacterium T-DNA terminal repeats.

6. The expression vector of claim 5, wherein the regulatory control elements comprise an alfalfa mosaic virus untranslated leader sequence, an endoplasmic reticulum retention signal, or both.

7. The expression vector of claim 5, wherein the rabies human MAb S057 subunits comprise a heavy chain, a light chain, or both.

8. The expression vector of claim 6, wherein the alfalfa mosaic virus untranslated leader sequence and endoplasmic reticulum retention signal were linked at the N- and C-terminus of the heavy chain, respectively.

9. The expression vector of claim 7, wherein the heavy chain, the light chain or both are under the control of one or more promoters.

10. The expression vector of claim 9, wherein the one or more promoters comprise one or more constitutive promoters.

11. The expression vector of claim 10, wherein the constitutive promoters comprise a cauliflower mosaic virus 35S promoter with duplicated upstream B domains, and a potato proteinase inhibitor II promoter.

12. The expression vector of claim 5, wherein the expression vector is pBIRA-57.

13. A host plant comprising the expression vector of claim 6.

14. The host plant of claim 13, wherein the expression vector is pBIRA-57 and the host plant is a tobacco plant.

15. The host plant of claim 13, wherein the plant comprises whole plant; plant cells, tissues, and organs.

16. A pharmaceutical composition for treating, ameliorating or preventing a rabies virus related disease or disorder comprising a pharmaceutically effective amount of a plant-derived human monoclonal antibody comprising a rabies MAb^{P}, and a pharmaceutically acceptable carrier or diluent.

17. The pharmaceutical composition of claim 16, wherein the rabies MAb^{P} comprises rabies human MAb^{P} S057.

18. A vaccine composition to induce passive immunity against rabies infection in humans comprising a rabies MAb^{P} S057, and an adjuvant.

19. A diagnostic test kit for detection of rabies infection comprising a rabies MAb^{P} and a detection agent comprising a detectable label.

21. A method of treating, ameliorating, or preventing a rabies related disease or disorder comprising, administering to an individual in need thereof an effective amount of the pharmaceutical composition of claim 11.

22. A plant-derived human monoclonal antibody comprising a a rabies MAb^{P}, wherein the a rabies MAb^{P} comprises an endoplasmic reticulum retention signal, and contains about 70% oligomannose type N-glycans.

23. The plant-derived human monoclonal antibody of claim 22, wherein the antibody contains about 70% Man6-9GlcNAc₂, about 3% GlcNAc₂Man₃GlcNAc₂, and about 3% GlcNAc₂(Xyl)Man₃GlcNAc₂.


SEQUENCE LISTING (8 sequences)

SEQ ID NO: 1 - DNA, 1431 bp, Homo sapiens
atggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcaacaggtatactgtcaactgggtgcgacaggcccctggacaagggcttgagtggatgggaggcatcatccctatctttggtacagcaaactacgcacagaggttccagggcagactcaccattaccgcggacgaatccacgagcacagcctacatggagctgagcagcctgagatctgatgacacggccgtgtatttctgtgcgagagagaatctcgataattcggggacttattattatttctcaggctggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 2 - protein, 477 aa, Homo sapiens
MDWTWRFLFVVAAATGVQSQVQLVQSGAEVKKPGSSVKVSCKASGGTFNRYTVNWVRQAPGQGLEWMGGIIPIFGTANYAQRFQGRLTITADESTSTAYMELSSLRSDDTAVYFCARENLDNSGTYYYFSGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKZ

SEQ ID NO: 3 - DNA, 729 bp, Homo sapiens
atgagtgtccccaccatggcctgggctctgctcctcctcagcctcctcactcagggcacaggatcctgggctcagtctgccctgactcagcctcgctcagtgtccgggtctcctggacagtcagtcaccatctcctgcactggaaccagcagtgatattggtggttataactttgtctcctggtaccaacaacacccaggcaaagcccccaaactcatgatttatgatgccactaagcggccctcaggggtccctgatcgcttctctggctccaagtctggcaacacggcctccctgaccatctctgggctccaggctgaggatgaggctgattattactgctgctcatatgcaggcgactacaccccgggcgtggttttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 4 - protein, 243 aa, Homo sapiens
MSVPTMAWALLLLSLLTQGTGSWAQSALTQPRSVSGSPGQSVTISCTGTSSDIGGYNFVSWYQQHPGKAPKLMIYDATKRPSGVPDRFSGSKSGNTASLTISGLQAEDEADYYCCSYAGDYTPGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSZ

SEQ ID NO: 5 - DNA, 25 bp, Artificial Sequence
  Primer for MAbS057 heavy chain DNA
cgccatggactggacctggaggttc

SEQ ID NO: 6 - DNA, 60 bp, Artificial Sequence
  Primer for MAbS057 heavy chain DNA
gctctagattagagctcatctttgtgatggtgatggtgatgtttacccggggacagggag

SEQ ID NO: 7 - DNA, 29 bp, Artificial Sequence
  Primer for MAbS057 light chain DNA
cgggatccatgagtgtccccaccatggcc

SEQ ID NO: 8 - DNA, 29 bp, Artificial Sequence
  Primer for MAbS057 light chain DNA
cgctgcagctatgaacattctgtaggggc